(19) United States
(12) Patent Application Publication
(10) Pub. No.: US 2011/0300069 A1
(43) Pub. Date: Dec. 8, 2011

(54) Methods of Using Labeled Ligands Having Human CD4 Specificity

(75) Inventors: Frank Emmrich, Leipzig (DE); Raimund W. Kinne, Jena (DE); Rudiger Laub, Leipzig (DE); Ullrich Pigla, Halle (DE)

(73) Assignee: BIOTECTID GMBH, Leipzig (DE)

(21) Appl. No.: 13/211,414

(22) Filed: Aug. 17, 2011

Related U.S. Application Data
(63) Continuation of application No. 10/498,633, filed on Dec. 20, 2004.

(30) Foreign Application Priority Data
Dec. 11, 2001 (DE) 101 60 653.2
Jul. 16, 2002 (DE) 102 32 189.2

(51) Int. Cl. A61K 51/10; A61K 49/00; C07K 1/107

(52) U.S. Cl. 424/1.49; 530/391.3; 424/9.1

(57) ABSTRACT

The invention concerns the use of a labelled ligand having specificity for the human CD4 molecule to produce a diagnostic agent for analysing migration and/or distribution patterns of certain cell populations which comprise CD4-bearing cells in human individuals.

In addition the invention concerns a composition which comprises a labelled ligand having specificity for the CD4 molecule and CD4-bearing cells or particles, and a method for determining the extent and progression of diseases in which human CD4-bearing cells are of clinical importance.

The invention concerns the use of a labelled ligand having specificity for the human CD4 molecule to produce a diagnostic agent for analysing migration and/or distribution patterns of certain cell populations which comprise CD4-bearing cells in human individuals.

The monomeric glycoprotein CD4 is the characteristic cell surface molecule of helper T lymphocytes. It has a molecular weight of 55 kDa and is composed of an extracellular region, a hydrophobic transmembranous part and a cytoplasmic part. Human CD4 is not only expressed on human helper T lymphocytes but also at a low density on T lymphocyte precursors in the bone marrow and in the thymus. In humans monocytes, macrophages, dendritic cells and eosinophilic granulocytes also carry the CD4 molecule [Marsh & Pelchen-Matthews, 1996]. The human CD4 gene is located on the short arm of chromosome 12 [Isobe et al., 1986].

Due to its four immunoglobulin-like extracellular domains (D1-D4), the CD4 molecule is classed as a member of the immunoglobulin superfamily [Haddon et al. 1985]. The amino-terminal domain 1 (D1) has a similar structure to a variable immunoglobulin domain. Together with domain 2 (D2) it forms a rigid structure of 60 Angströms in length. Domains D1 and D2 are connected by a flexible joint to domains D3 and D4.

The most important natural CD4 ligands are class II molecules of the major histocompatibility complex (MHC) on so-called antigen-presenting cells (APC). These are specialized cells of the immune system which prepare antigenic structures and make them recognizable for immune responses. The extracellular domains D1 and D2 of the CD4 molecule mainly associate with non-polymorphic regions of the 2 chain of the MHC class II molecule [Clayton et al., 1989; Cammarota et al. 1992]. The a2 chain of the MHC class II molecule (MHC-II) may also be significant for the interaction with CD4 [Vignali et al., 1996].

CD4 molecules can associate with one another via their D1 domains to form functional oligomers which stabilize the T cell receptor (TCR)/MHC II peptide complex and increase the efficiency of signal transduction [Li et al., 1998]. CD4 can also associate with the TCR/CD3 complex via the proximal domains D3 and D4 [Vignali et al., 1996]. The CD4 molecule enhances the antigen-specific stimulation of T cells during interaction of the TCR with the antigen-peptide-presenting MHC-II. Hence the CD4 coreceptor system not only enables an increase in antigen sensitivity but also the maintenance of a high antigen specificity [Konig et al. 1996].

CD4 is actively involved in signal transduction in T lymphocytes. Its cytoplasmic domain is non-covalently associated with a T cell-specific protein tyrosine kinase (p56lck) of the so-called scr family [Veilette et al., 1988]. The proximal part of the cytoplasmic CD4 domain interacts via cysteine residues with the N-terminal region of p56lck [Turner et al., 1990].

Other natural CD4 ligands for human CD4 are the gp120 of the human immuno-deficiency virus (HIV), the chemotactic cytokine IL-16 and immunoglobulins. The binding sites for gp120 and MHC-II are not identical but are in close proximity and partially overlap. HIV gp120 mainly binds to the D1 domain of the CD4 molecule [Clayton et al. 1989]. Infection of CD4+ cells with HIV results in a loss of CD4 expression on the cell surface [Hoxie et al., 1986a]. This limits the ability of infected cells to interact with MHC-II of the APC and favours the progression of the disease.

Chronic and chronic-recurrent inflammations occur in association with many diseases in which the immune system is of importance. Different organs and tissues may be affected by chronic recurrent inflammations. In contrast to acute inflammations, the cell types that accumulate in the inflammatory foci which are fundamentally qualitatively different. They can be identified on the basis of specific surface molecules. One of these molecules is the human CD4 molecule which is expressed at different levels on, among others; helper T lymphocytes, monocytes, macrophages, dendritic cells and eosinophilic granulocytes. These cells and in particular the T lymphocytes are an important population of chronic inflammatory cells.

Certain monoclonal antibodies can recognize the human CD4 molecule highly specifically, bind to its protein structure and thus highly specifically bind human-CD4-expressing cells in inflammation.

Under physiological conditions these inflammatory cells are present in an inactive differentiation state and follow characteristic migration and/or distribution patterns in primary and secondary lymphatic organs and in the circulating blood.

Furthermore tumour cells can develop from human CD4-bearing cells which can result in the development of malignant or benign tumour diseases. In certain tumour diseases e.g. in certain lymphomas, the expression of the human CD4 molecule may be preserved.

Hence the qualitative and quantitative detection of CD4-expressing cells is an established immunodiagnostic agent in vitro method. Whereas flow cytometric analysis allows their quantity to be detected in peripheral blood, immunohistological methods allow their local distribution pattern to be analysed ex vivo i.e. after biopsy of suspect tissue.

However, both methods are not able to give objective information on the migration and/or distribution patterns of T helper cells in the living organism or on their possible accumulation and their extent in diseased tissue/organs. This is due to the following reasons:
(1) Peripheral blood contains a maximum proportion of 5% of all human CD4-expressing cells of the body which is only representative to a limited extent;
(2) in each individual there is considerable variation in the numbers of peripheral human CD4-expressing cells from day to day;
(3) the peripheral human CD4-expressing cell counts do not in any way allow the identification and localization of local accumulations in chronic inflammatory foci or the location of degenerate human CD4-expressing cells in tumours,
(4) the probability that a biopsy will capture the decisive region for diagnosis of an inflammatory focus or tumour or a metastasis is very small;
(5) from the biopsy material it is not possible to make any conclusions about the actual spatial dimensions of the inflammatory region or of the tumour or of the metastasis; and
(6) follow-up and therapeutic monitoring can only be achieved by repeated biopsies and is a considerable burden for the affected individual.

U.S. Pat. No. 6,146,614 describes a method for determining lymphocyte distribution and migration in mammals using imaging methods and the use of these determinations to diagnose the course of various diseases and to monitor the response to various treatments for such diseases and to identify drugs for treating these diseases. The examples of U.S. Pat. No. 6,146,614 concern the use of monoclonal antibodies to mouse CD4 antigen. One example concerns the use of such antibody to label human cells. However, this example contradicts the generally known fact that there is no cross-reactivity between human CD4 and murine CD4. Hence example 7 of the present application shows a corresponding comparative experiment which clearly shows that the antibody to murine CD4 described in U.S. Pat. No. 6,146,614 has no cross-reactions with human CD4. Thus the method described in U.S. Pat. No. 6,146,614 is not suitable for determining the distribution and migration pattern of CD4-bearing cells in human individuals.

Due to the limitations of conventional diagnostic methods described above there is an urgent need for a method for the non-invasive determination of the migration and/or distribution pattern and accumulation of inflammatory cells and tumour cells which carry CD4 surface molecules as well as their redistribution and dissolution under therapeutic conditions (therapeutic monitoring).

Hence the object of the invention is to overcome the aforementioned disadvantages of the prior art. In particular it is an object of the invention to enable the determination of the migration and/or distribution pattern of human CD4-expressing cells in human individuals while avoiding invasive diagnostic measures.

Another object of the invention is to reliably, effectively and simply determine the migration and/or distribution pattern of human CD4-expressing cells in patients with a (suspected) diagnosis of chronic inflammatory diseases, infectious diseases or tumour diseases or the course of an immunosuppressive treatment after a transplantation in human individuals.

Another object of the invention is the differentiation of chronic inflammatory foci such as in autoimmune diseases and acute inflammations such as inflammations due to infection based on the finding that more cells with the said surface antigen are present in chronic inflammatory infiltrates than in acute inflammatory infiltrates. Thus an increased number of CD4-expressing cells can be detected in the chronically inflamed synovial membrane in rheumatoid arthritis. In this case the CD4/CD8 ratio is 2- to 7-fold higher than that of peripheral blood [Immunology of rheumatic diseases. Publ. S. Gupta & N. Talal. Plenum Medical Book Company, New York and London, 1985]. Moreover, the antigen-specific activation of helper T lymphocytes results in a considerable increase of CD4 surface expression [Ridgeway et al., 1998, J. Immunology 161:714-720].

Another object of the invention is to enable the localization and determination of the spatial size and intensity of inflammatory foci in autoimmune diseases and an assessment of the course of autoimmune diseases or influence of therapeutic measures on autoimmune diseases.

Another object of the invention is to reliably, effectively and simply localize tumours or metastases thereof which express human CD4 in human individuals with a (suspected) diagnosis of malignant or benign diseases of the haematopoietic system, to determine the spatial size of human CD4-bearing tumours or metastases thereof and to assess the progress of tumour diseases (follow-up) and therapeutic measures for tumour diseases (therapeutic monitoring).

According to the invention these objects are achieved by using a labelled ligand having specificity for the human CD4 molecule to produce a diagnostic agent to analyse migration and/or distribution patterns of certain cell populations which comprise human CD4-bearing cells in human individuals.

The spatial position or arrangement of human CD4-bearing cells in cell populations in the human organism is referred to as a distribution pattern in the sense of the present invention. The position or arrangement of human CD4-expressing cells relates to their distribution in the body's own tissues or in introduced foreign tissues and synthetic materials or their arrangement at the interfaces of the said foreign tissue and synthetic materials. The distribution of human CD4-bearing particles develops after they have been administered into the human organism. The distribution pattern can be determined for the entire organism as well as for parts of the organism.

For human CD4-bearing particles, the human CD4 molecule can be produced in recombinant production systems (recCD4). Suitable carriers of different sizes can then be coated with recCD4. Carrier materials can be plastic particles and also structures that are enclosed by membranes and contain therapeutically active substances in their interior. Thus the human CD4 molecule can be attached as a recognition structure (guide) to carriers or particles or vesicles such as liposomes in order to bring them, for example filled with pharmacological substances, to target cells which in turn carry molecules that interact with CD4. These molecules are members of the MHC class II antigens (in the case of humans, HLA class II antigens) that are expressed on various body cells such as monocytes, macrophages, dendritic cells, B cells and can be upregulated on a number of other cells by the action of factors in their surroundings such as certain cytokines. Since the upregulation of MHC class II molecules has been linked to pathogenic processes, this enables human CD4-guided pharmacologically active substances to be directed into certain tissue zones. CD4-bearing particles can for example be used to locate complementary structures e.g. for the surface protein gp120 of HIV and mediate a local accumulation of antiviral agents. It is, however, also conceivable to bind anti-CD4 antibodies or antibody fragments as molecular guides to particles, molecules or vesicles which then can be directed into CD4-bearing tissue zones.

A migration pattern of human CD4-bearing cells or particles is understood in the sense of the present invention as the movement of human CD4-bearing cells or particles in the human organism including its tissues and peripheral blood. The migration of human CD4-bearing cells or particles can be determined for the entire organism or for certain parts or organs thereof.

Human CD4-expressing cells in the sense of the present invention can include monocytes, macrophages, dendritic and Langerhans cells, eosinophilic granulocytes and helper T lymphocytes. In certain cases these cells are contained in the composition of inflammatory infiltrates in autoimmune diseases, infectious diseases, malignant diseases or in transplantations or implantations. In other cases the regulation of these cells is altered (degenerate) so that they form tumours. Other cases concern complications after transplantation of organs or tissues or after implantation of synthetic substitutes. These are complications that can result in the loss of the transplantate or implantate (immunological rejection reactions).

A substance or compound which can specifically interact with CD4-bearing cells is referred to as a ligand in the sense of the present invention. A ligand can for example interact with certain molecules that are expressed on the surface of cells or are attached to the surface of particles and in this case with CD4 molecules. The ligand can be any substance or compound that has the property of being able to exclusively or predominantly interact with the CD4 surface molecule and bind to this molecule.

The diagnostic agent that can be used according to the invention comprises a labelled high affinity ligand having specificity for the human CD4 molecule.

The labelled ligand is preferably selected from the group consisting of antibodies, antibody fragments, recombinant antibodies, recombinant antibody fragments, synthetic peptides having a high affinity for the CD4 surface antigen, peptido-mimetics, carbohydrates and glycoproteins. The labelled ligand particularly preferably recognizes an epitope in the D1 domain of the human CD4 molecule.

In particular antibodies, antibody fragments, recombinant antibodies and/or recombinant antibody fragments are preferred of which monoclonal antibodies or monoclonal antibody fragments or antibodies or antibody fragments derived therefrom are especially preferred. Furthermore, the antibodies, antibody fragments, recombinant antibodies or recombinant antibody fragments can preferably recognize one of the sequences shown in SEQ ID NO.1 to SEQ ID NO.6 and bind thereto. The antibodies, antibody fragments, recombinant antibodies or recombinant antibody fragments particularly preferably recognize the conformation epitope (FIG. 2) of human CD4 and bind thereto. The antibodies, antibody fragments, recombinant antibodies or recombinant antibody fragments can be anti-CD4 antibodies. In particular antibody fragments can be particularly preferably used according to the invention.

Ligands can be polyclonal or monoclonal antibodies of which monoclonal antibodies are preferred and in particular the monoclonal antibody Max.16H5. However, whole antibodies may be immunogenic and induce immune reactions directed towards themselves after administration to a human organism. Since smaller ligands are less immunogenic, these are preferred ligands.

Antibody fragments such as F(ab′)2, Fab′ or Fab are preferred as smaller ligands. The anti-human CD4 Fab′ fragment is particularly preferred and in particular that of the monoclonal antibody Max.16H5.

An example of a smaller ligand is OKT3 (Orthoclone). Orthoclone is a monoclonal anti-human CD3 antibody which is formed by mouse hybridoma cells and is used by Ortho Pharmaceutical for example to treat immunological rejection reactions after transplantations. Other monoclonal antibodies or antibody fragments e.g. against human CD4 can be produced in a known warmer using the hybridoma technique.

Those antibodies or antibody fragments can be used as the ligand which are produced by recombinant methods in microorganisms such as single-chain antibodies, for example single chain Fv (scFv), antibody-like structures such as minibodies and other fragments that can bind specifically to a surface molecule such as human CD4 (human CD4) for example recombinant antibodies.

Proteins, glycoproteins, peptides, peptidomimetics, aliphatic and cyclic hydrocarbon compounds and aptamers can also be used as ligands which bind with a high specificity to a surface molecule such as CD4. Peptidomimetic refers to a compound which is not a peptide but is similar to a peptide with respect to important properties such as size and charge distribution. An aptamer is a DNA or RNA structure having a high affinity for the said surface molecules e.g. for human CD4.

The ligand can be labelled by any labelling molecule that enables the labelled ligand to be localized in time and space with medical imaging methods.

The labelled ligand is preferably selected from the group consisting of gamma emitters, positron emitters, magnetic material, density contrast material and mixtures thereof.

The labelled ligand can be a gamma emitter which comprises one or more radioisotopes selected from the group consisting of indium-111, technetium-99m, technetium-99, iodine-132, other radioisotopes for medical imaging methods and mixtures thereof. Technetium-99m and indium-111 are particularly preferred as radioisotopes.

In another preferred embodiment the labelled ligand is a positron emitter which comprises one or more isotopes selected from the group consisting of fluorine-18, carbon-11, iodine-124 other isotopes for medical imaging methods and mixtures thereof.

In another preferred embodiment the labelled ligand can comprise a magnetic material selected from the group consisting of paramagnetic substances such as gadolinium, superparamagnetic substances, hydrated iron oxide particles, other materials for medical imaging methods and mixtures thereof.

The labelled ligand can also comprise a density contrast material.

The human CD4-bearing cells are preferably selected from the group consisting of T and B lymphocytes, monocytes, macrophages, dendritic and Langerhans cells and eosinophilic granulocytes and in particular the lymphocytes can be selected from a group or population that comprises non-lymphocytic cells.

The migration and/or distribution pattern of cell populations that comprise human CD4-bearing cells can be analysed by means of the specific interaction of the diagnostic agent with CD4-bearing cells after administration of the labelled ligand to human individuals. The specific interaction of the in vivo diagnostic agent results in labelled cells. These cells are substantially involved in the inflammatory infiltrate especially in chronic inflammations.

Alternatively the migration and distribution pattern of cell populations that comprise human CD4-bearing cells can be analysed outside the human organism by specific interaction with cell populations that are derived from a human organism and contain CD4-bearing cells. In this case the specific interaction also results in labelled cells that are introduced into an organism. Labelling of cells outside the body for use in the same patient is common practice is nuclear medicine. However, previously cells have been labelled directly in this manner with various isotopes. This is either for technical reasons (high concentration of label required), to protect the health of the patient (unbound radioactivity is removed before it is returned to the patient) or/and for economic reasons (the valuable products are used effectively for labelling in very small volumes).

Labelling outside the human organism can be carried out as follows: Blood is withdrawn from the patient. Cells are prepared from this blood and incubated outside the body with isotope-labelled ligands; e.g. anti-CD4 antibodies. Afterwards unbound ligand is removed and the purified cell preparation is again injected into the same patient. Then the distribution of the labelled cells in the body is monitored with known and described methods (gamma camera or detector probe). The use of this method is for example of interest when labelled cells are injected into tumours or into the vicinity of tumours during an operation. They then search for the outflowing lymph vessels and the downstream lymph nodes. In this manner a pencil-like detector probe can be used even during the operation to determine into which lymph nodes these cells have preferentially flowed. These particularly frequented lymph nodes are also the probable sites of metastasis and hence the surgeon can immediately remove them.

The use according to the invention of the diagnostic agent enables the migration and/or distribution pattern of cells which carry CD4 surface molecules to be determined in the organism with the aid of medical imaging methods. The ability to visualize migration and/or distribution patterns under normal and pathological conditions allows the extent and progression of diseases for which the said cells are of clinical importance to be determined.

In particular analysis of the migration and/or distribution pattern of human CD4-bearing cells with the aid of the inventive use of the diagnostic agent enables chronic inflammatory foci to be localized in the human organism and differentiated from acute inflammations. Furthermore, analysis of the migration and/or distribution patterns of human CD4-bearing cells is also suitable for clarifying tentative diagnoses, for monitoring the clinical course and for monitoring the response to anti-inflammatory treatments.

Another field of application for analysing migration and/or distribution patterns of human CD4-bearing cells is to find malignant cells (tumour cells) on which the said surface molecules are expressed and which interact with the diagnostic agent such that labelled tumour cells are formed. In particular the analysis of the migration and/or distribution patterns of human CD4-bearing cells can be used to locate tumours of the haematopoietic system or of metastases thereof provided the said surface molecule is expressed on them.

In addition the diagnostic agent can also be used to carry out a so-called radioimmunotherapy (RIT). For RIT the diagnostic radioisotopes are replaced by those with a strong α or/and β radiation such as Re-186, Re-188, I-131, Y-90, Sm-153 as β emitters and Bi-213 as an α emitter. Similarly derivatized antibodies, fragments thereof or ligands are also used. In the case of DTPA-derivatized molecules these are for example In-111 and Y-90. I-123 and 1-131 as well as Tc-99 and Re-188 are additional isotope pairs for diagnosis and therapy. The α or/and β radiation emitted by radioisotopes that are used therapeutically destroys cells that are in the immediate vicinity of the radioimmunotherapeutic agent or have been labelled by this agent. In this manner it is possible to selectively destroy labelled tumour cells or harmful inflammatory cells or other undesired cells. An advantage in this case is that not only the cell carrying the CD4 molecule is damaged or destroyed but also cells that are in its immediate vicinity. More remote tissue is not affected.

Furthermore the diagnostic agents according to the invention can also be used to visualize and quantify inflammatory cells in infectious diseases e.g. viral diseases. Hence they also enable assessments of stages, follow-up and treatment monitoring e.g. of swollen lymph nodes, certain tumour diseases and monitoring antiviral treatments.

In particular the use according to the invention for analysing migration and distribution patterns of certain cell populations in human individuals in diseases or tentative diagnoses is suitable for diseases of the following types:

Autoimmune diseases, infectious diseases, malignant and benign diseases of the haematopoietic system, infectious diseases or diseases for which either CD4-bearing or CD8-bearing T lymphocytes are assumed to be of pathogenetic importance.

Autoimmune diseases may be rheumatoid arthritis, multiple sclerosis, systemic lupus erythematosus, psoriasis or an inflammatory intestinal disease such as Crohn's disease or ulcerative colitis. Malignant or benign diseases may be diseases of the haematopoietic system such as myeloma, lymphoma or leukaemia or solid tumours such as melanoma, kidney cell carcinoma, ovarial carcinoma or lung carcinoma. Infectious diseases may be all diseases that are caused by bacteria or viruses such as HIV.

The use according to the invention of the labelled ligand as a diagnostic agent can also serve to determine the distribution and/or migration patterns of CD4-bearing cells when one or more transplantates are present in a human individual such as cells, tissues or organs and/or implantates such as tissue or vessel substitutes, depots, pumps or filters.

The migration and/or distribution pattern is preferably determined by medical imaging methods such as radioimaging, magnetic resonance methods or optionally by computer-assisted tomographic imaging methods. The imaging can either consist of single images or a series of images. The image may be made of the whole individual or parts thereof (scanning).

The migration and/or distribution pattern of a ligand labelled with a gamma emitter can be determined with a gamma camera or a camera for single photon emission tomography (SPECT). Positron emitters can for example be determined by positron emission tomography (PET). The migration and/or distribution pattern of magnetic particles in the organism can be determined by magnetic resonance tomography (MRT) or magnetic resonance imaging (MRI). The use and handling of these medical imaging methods are known.

The medical imaging method is preferably a whole body analysis (scan). The invention also encompasses scans of individual body parts or organs. The scans can be carried out minutes, hours, days, weeks or longer after administration of the labelled ligand. The exact time depends on various factors e.g. on the type and amount of the labelled ligand, on the behaviour of the human CD4-bearing cells or particles or on disease courses. The exact time factor can be determined by experienced technical personnel with the aid of commonly used optimization algorithms. The medical imaging method is completed with single or serial scans.

The use according to the invention of the labelled ligand as a diagnostic agent to analyse the migration and/or distribution patterns of CD4-bearing cells can also be used to determine the therapeutic response or efficiency of a treatment in a human individual who suffers from one or more of the said diseases. The individual is treated with a therapy for the corresponding disease(s). The migration and/or distribution pattern of CD4-bearing cells in the treated individual as a reaction to the treatment is determined with medical imaging methods. This is carried out by determining whether the treatment has changed the distribution and/or migration pattern of human CD4-expressing cells.

The use according to the invention of the labelled ligand as a diagnostic agent to analyse the migration and/or distribution patterns of CD4-bearing cells can also be used to estimate the potential of a compound or of a drug to change the distribution and/or migration pattern of human CD4-expressing cells in the human organism by determining whether the compound or the drug has changed the distribution and/or migration pattern of human CD4-expressing cells in the human organism with the aid of medical imaging methods as described above. The distribution and/or migration patterns are changed when they deviate from a standardized distribution and/or migration pattern.

The use according to the invention of the labelled ligand as a diagnostic agent to analyse the migration and/or distribution pattern of CD4-bearing cells can also be used to identify drugs for treating autoimmune diseases, tumour diseases or infectious diseases by determining whether the compound or the drug changes the distribution and/or migration pattern of human CD4-expressing cells in the human organism. The distribution and/or migration pattern obtained by administering the compound or the drug is compared with that of human individuals who do not suffer from one of the said diseases.

The use according to the invention of the labelled ligand as a diagnostic agent to analyse the migration and/or distribution patterns of CD4-bearing cells also allows the determination of an optimal protocol for carrying out an immunosuppressive therapy after transplantation of non-autologous cells, tissues or organs or cells, tissues or organs which, for other reasons, may be potentially immunogenic for the recipient and after implantation of synthetic and semi-synthetic materials as a substitute for or to assist tissues and organs and their functions by using medical-imaging methods as described above to determine whether the distribution and/or migration patterns of human CD4-bearing cells have changed in such a manner that it indicates an inflammatory reaction or immune reaction associated with the transplantate/implantate. This conclusion can either be based on a deviation of the said distribution and/or migration pattern from standardized patterns or a deviation from patterns that have been determined for the said individual at an earlier time.

Another subject matter of the present invention is a composition which comprises a labelled ligand having specificity for the CD4 molecule and CD4-bearing cells or particles. The CD4-bearing cells or particles are preferably human CD4-bearing cells or particles.

The composition is preferably a therapeutic or diagnostic agent and the composition is particularly preferably a diagnostic agent for determining the distribution and migration pattern of CD4-bearing cells or particles in an individual, preferably in a human individual.

The labelled ligand and the CD4-bearing cells are preferably as described above. The particles are preferably selected from spherical particles of various materials. The particles can for example be liposomes. The CD4-bearing particles can interact with complementary structures for example on body cells, bacteria or viruses, label the same or transport active substances into their vicinity. The particles may also contain a drag, preferably a diagnostic or therapeutic agent.

The composition according to the invention and in particular the diagnostic agent is suitable for determining the migration of human CD4-expressing cells or synthetic particles in human individuals. For this purpose CD4-bearing cells such as inflammatory cells, or particles containing the labelled ligand are incubated such that a composition according to the invention comprising labelled cells or particles is obtained. This composition and in particular the diagnostic agent comprising labelled cells or particles can be administered to a patient and their distribution and migration pattern can be determined as described above by medical imaging methods.

The composition according to the invention and in particular the diagnostic agent can be produced by contacting the labelled ligand with the CD4-bearing cells and/or particles. The CD4-bearing cells can either be derived from the same human individual in which the migration and/or distribution pattern of CD4-bearing cells is to be determined, or be derived from another human individual. The preparation of the composition and in particular of the diagnostic agent can be carried out in vitro by means of a specific interaction of a labelled ligand with CD4-bearing cells or particles to form labelled human CD4-bearing cells or particles. If the composition according to the invention comprises CD4-bearing cells, the human CD4-bearing cells advantageously do not have to be separated or isolated in order to specifically label them with the ligand. They can for example be present in a cell population. Such a cell population can be incubated with the labelled ligand in order to label the human CD4-bearing cells. For example peripheral blood can be incubated with the labelled ligand to obtain a diagnostic agent according to the invention. The labelled ligand interacts with human CD4-bearing cells or particles to form specifically labelled cells or particles. In this connection interact means bind, associate, complex or conjugate.

Administration of the labelled ligand to a human individual can be carried out by all methods that enable it to specifically interact with human CD4-bearing cells or particles in the human organism to form labelled human CD4-bearing cells or particles. These methods comprise injection, infusion, incorporation into depots, implantation, oral ingestion and rectal administration as well as local application. The preferred method of administration is injection which can for example be carried out intravenously, intradermally, subcutaneously, intramuscularly or intraperitoneally. In certain cases multiple or combined administration methods are used. The labelled ligand may be present in a dissolved or colloidal form in which case the carrier liquids for administration in human organisms are suitable such as water or physiological saline for an injection.

The diagnostic agent according to the invention can be used at a dose that is preferably between 0.01 mg/kg and 7 mg/kg body weight. If the ligand is an antibody, the dose is preferably between 0.2 mg/examination and 7 mg/examination or between 0.003 mg/kg and 0.1 mg/kg body weight. The amount of required radioactivity depends on the isotope that is used and can be determined by experienced technical personnel. If the ligand is labelled with a radioactive isotope, the activity can be between 1.5 and 10.0 mCi per single dose for indium-111, between 10 and 30 mCi per single dose for technetium-99, between 5 and 10 mCi per single dose for iodine-123, between 5 and 10 mCi per single dose for iodine-124, between 10 and 20 mCi per single dose for fluorine-18 and between 20 and 30 mCi per single dose for carbon-11.

If the diagnostic agent comprises particles carrying human CD4, it can be prepared in vitro by specifically interacting a labelled ligand as described above with the particles to obtain a diagnostic agent according to the invention comprising labelled human CD4-bearing particles.

The labelled human CD4-bearing particles can be administered to the human organism by various methods as described above in relation to the diagnostic agent according to the invention comprising CD4-labelled cells and labelled ligands.

The determination of the migration and/or distribution pattern of the composition according to the invention and in particular of the diagnostic agent according to the invention can be advantageously used in the same fields of application as described above in relation to the use according to the invention of the labelled ligands having specificity for the human CD4 molecule:

Another aspect of the present invention concerns a method for determining the extent and progression of diseases in which human CD4-bearing cells are of clinical importance comprising the steps:

a) providing an in vivo analysis of the distribution and/or migration pattern of human CD4-bearing cells in an individual,
b) providing a standard in vivo analysis of the distribution and/or migration pattern of human CD4-bearing cells in an individual and
c) determining deviations of the analyses provided in step a) and step b) thus enabling the extent and progression of the disease to be determined.

The analysis provided in step a) and/or step b) is preferably obtained by administering a labelled ligand or the diagnostic agent according to the invention to an individual as described above and determining the distribution and/or migration pattern of CD4-bearing cells or of the diagnostic agent in the individual using medical imaging methods as also described above.

The analysis provided in step a) is preferably of a diseased human individual. The disease is preferably selected from the group consisting of autoimmune diseases, tumour diseases, infectious diseases and rejection crises after transplantations as described above.

The standard analysis in step b) is preferably the analysis of the distribution and/or migration pattern of human CD4-bearing cells in a non-diseased individual.

In a further preferred embodiment the standard analysis in step b) is a distribution and/or migration pattern of an individual that is obtained from a previous analysis of the same human individual.

In step a) the analysis of the distribution and/or migration pattern of human CD4-bearing cells in an individual can also be provided in an individual who has additionally been treated for the disease. The treatment is preferably administration of a compound or drug. The response of the individual to the therapy is characterized by using medical imaging methods to determine whether the treatment has changed the distribution and/or migration pattern of human CD4-bearing cells. These patterns are preferably compared with those of the diseased individual for example before the start of treatment or at an earlier time in the treatment or with those of non-diseased individuals.

The invention is illustrated in the following on the basis of figures and examples.

### FIGURES

FIG. 1 shows dot blots of a specific staining of murine and human helper T lymphocytes: The dot blots represent the following double staining: A. mouse cells: murine CD3/murine CD4 (GK 1.5); B. human cells: human CD3/human CD4 (max. 16H5); C. mouse cells: murine CD3/human CD4 (max. 16H5); D. human cells: human CD3/murine CD4 (CK 1.5).

FIG. 2 shows the conformation isotope of human CD4 in the calorie model.

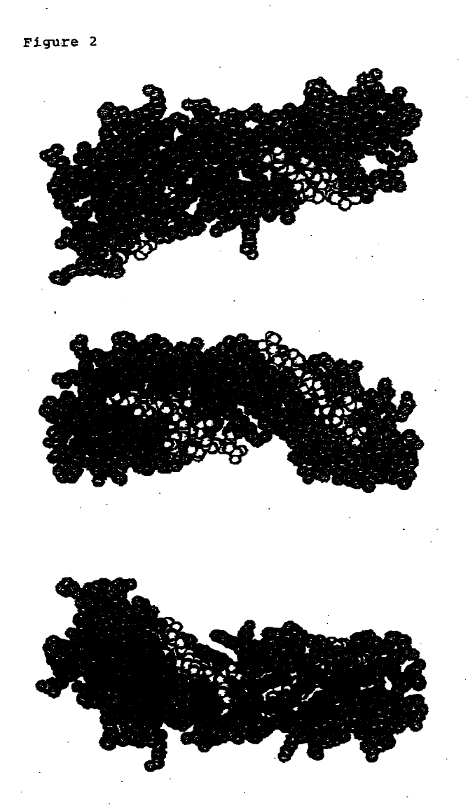

### EXAMPLES

### Example 1

### Production of Anti-Human CD4 and Enzymatic Cleavage to Form Fab′ Fragments

The example describes the production and purification of a monoclonal mouse antibody having specificity for human CD4 and its enzymatic cleavage into Fab′ fragments.

The monoclonal antibody Max.16H5 recognizes an epitope in the D1 domain of human CD4. The immuno globulin (IgG1) was isolated from hybridoma supernatants by means of protein A affinity chromatography. The eluate from the protein A column was neutralized, dialysed against PBS and the immunoglobulin concentration was adjusted to 1 mg/ml. The identity and specificity of the monoclonal antibody was shown by SDS gel electrophoresis using mouse IgG1 as a control and in a Western blot using recombinant human CD4. Binding to natively expressed human CD4 (bioactivity) was detected by flow cytometry (FACS) using a FACScan (Becton-Dickinson). Cryopreserved human peripheral mononuclear cells (PBMC) which contain an (individually different) proportion of human CD4-expressing helper T lymphocytes were used as indicator cells. After thawing, they were incubated with anti-human CD4, washed and bound mouse IgG was quantitatively determined in a FACScan using fluorescently (FITC)-labelled goat anti-mouse immunoglobulin Fab2 fragments (DAKO F 0479).

Anti-CD4 total IgG was digested with papain into Fab′ and Fe fragments. The enzymatic cleavage was terminated at previously optimized reaction times and the reaction mixture was fractionated by column chromatography on protein A. Fc fragments are bound in this process to protein A and mainly Fab′ fragments appear in the eluate. The size (ca. 50 kD) and proportion (ca. 90%) of the eluted Fab′ fraction was determined by SDS gel electrophoresis. The bioactivity of the anti-human CD4 Fab′ fraction i.e. its ability to selectively bind to natively expressed human CD4 was quantitatively determined by FACS analyses. For this human PBMC were successively incubated with Fab′ fragments and FITC-labelled goat anti-mouse immunoglobulin and the human CD4-expressing cells were analysed in a flow cytometer.

### Example 2

### Radiochemical Labelling of Anti-Human CD4 Fab′ Fragments with 111-In

The example describes the derivatization of anti-human CD4 Fab′ fragments with a chelating agent.

Anti-human CD4 Fab′ is reacted in 0.1 M NaHCO₃ buffer via an anhydride reaction with cyclic diethylenetriamine pentaacetic acid (cDTPA). The Fab′-DTPA derivative is then transferred into 0.5 M sodium acetate buffer pH 5.4, concentrated over a 10 kD exclusion filter and purified by size exclusion chromatography. Aliquots of the derivatized fragments are adjusted to a protein concentration between 0.5 and 1 mg/ml and stored at +4° C. until the radiochemical labelling.

The radiodiagnostic agent is prepared immediately before injection by labelling the anti-human CD4 Fab′-DTPA derivative with 111-indium (half-life: 2.6 days, medium-energetic gamma radiation of ca. 247 keV). For this about 10 g DTPA-derivatized fragment is incubated for 30 min at room temperature with 300 Ci 111-indium chloride. Free indium chloride is separated over PD-10 columns from the radiodiagnostic agent (111-indium-labelled anti-human-CD4 Fab′).

### Example 3

### Binding Assay with Anti-CD4 Fab′-DTPA

The example demonstrates that the specificity and bioactivity of anti-CD4 Fab′ from example 1 and DTPA-conjugated anti-CD4 Fab′-DTPA from example 2 are not different.

Indicator cells were incubated with stepwise increasing concentrations of Fab′ and DTPA-derivatized Fab′ of anti-human CD4 and unbound ligand was washed out with phosphate-buffered sodium chloride solution (PBS). The ligands bound to the indicator cells (human PBMC) were incubated with fluorescently (FITC)-labelled goat-anti-mouse immunoglobulin Fab2 fragments (DAKOF0479), washed again and the fluorescence of human CD4-expressing cells was quantitatively determined in a FACS.

The specificity of the anti-CD4 Fab′ fragments (for human CD4) is not influenced by the derivatization with DTPA. DTPA-derivatized and non-derivatized anti-CD4 Fab′ fragments identify fractions of fluorescent human CD4-bearing indicator cells of equal size. The biological activity of both ligands was comparable, saturation for the fluorescent staining was achieved at the same concentrations of Fab′ and Fab′-DTPA. Also the loss in fluorescence with decreasing concentrations of the ligands was comparable.

### Example 4

### Biodistribution of 111-Indium-Labelled Anti-Human CD4-Fab′ Fragments

The example shows that 111-indium-labelled anti-human CD4-Fab-DTPA (radiodiagnostic agent) selectively accumulates in tissues having a high expression of human CD4.

A mouse model with transgenic expression of human CD4 (CD4/DR3 mouse) was used as a preclinical model for examining the biodistribution of the radiodiagnostic agent in vivo. Human CD4 is expressed instead of the endogenous (wild-type) CD4 in the CD4/DR3 mouse model and the expression pattern of human CD4 correctly corresponds to the natural distribution of the receptor [Laub 2001].

20 Ci of the radiodiagnostic agent was injected intravenously into each of the CD4/DR3 mice and wild-type mice (C57/BL6): Lymphatic and non-lymphatic organs were removed 1, 4, 24 and 48 hours after injection, weighed and the incorporated radioactivity was measured with a gamma counter (LKB model 1282, Wallac-Perkin Elmer, Turku Finland). In order to correct for the half-life, aliquots of the radiodiagnostic agent were retained and measured at the same time as the samples. For the analysis of biodistribution, the radioactive signals (counts) were converted into % injected activity per gram tissue.

Significantly increased activities were measured in the lymph nodes, spleen and bone marrow in CD4/DR3 mice. The 111-indium-labelled anti-human CD4 Fab′ fragment did not accumulate in non-lymphatic organs (liver, muscle, lung and heart) [Laub 2000]. Signals were detected in the kidney which indicate a renal elimination of the radiodiagnostic agent.

The investigations on biodistribution show that the distribution of the radiodiagnostic agent in lymphatic tissue of the CD4/DR3 mouse is due to a specific interaction between the transgenically-expressed human CD4 and the 111-indium-labelled anti-human CD4 Fab′.

### Example 5

### Dependence of the Specific Accumulation on the Expression of Human CD4

This example shows that specific signals of the radiodiagnostic agent require the presence of human CD4.

20 Ci of the radiodiagnostic agent from example 2 was intravenously injected into Balb/c mice (wild-type) having a normal expression of mouse CD4. Samples were taken as described in example 4 from three Balb/c mice in each case after 1, 4, 24 and 48 hours and the incorporated radioactivity was measured. Elevated signals were only determined in the kidneys at all times which indicates a renal elimination of the radiodiagnostic agent or of the radionuclide. Although lymphatic organs such as spleen, lymph nodes and bone marrow contain up to 20% mouse CD4-expressing cells, no signals were detected here. The result shows that the 111-indium-labelled anti-human CD4 Fab′ specifically binds human CD4 and does not unspecifically accumulate in lymphatic organs of the mouse.

### Example 6

### In Vivo Localization of Helper T Lymphocytes in a Mouse Model with Transgenic Expression of Human CD4

The example shows that the radiodiagnostic agent enables organs and tissues with a high proportion of human CD4-expressing cells to be localized in vivo.

20 Ci of the radiodiagnostic agent was injected in each case into the tail vein of wild-type mice and transgenic CD4/DR3 mice which express human CD4 instead of the wild-type CD4. The animals were anaesthetised 1, 24 and 48 hours after the injection and while lying prone they were positioned in the measuring field of a gamma camera (Siemens Icon). The signals of the gamma photons were recorded for 30 min with the aid of a planar collimetor for medium energetic gamma radiation and compiled to form planar whole body scintigrams. Further evaluation of the scintigrams was carried out using a software for medical imaging methods (Osiris, University Hospital, Geneva, Switzerland).

One hour after injection of the radiodiagnostic agent particularly strong signals were detected in the kidneys in the mice carrying human CD4 and in wild-type mice. In wild-type mice a background persists that can be less differentiated and the kidney signals remain over the entire measurement period which indicates a renal elimination of the radiodiagnostic agent without there being specific accumulations in other organs. In CD4/DR3 mice which transgenically express human CD4 there are also signals in the area of the spleen in addition to the kidney signals four hours after injection. Clear signals are also measured in the area of the front and rear extremities which are derived from human CD4-expressing cells in the bone marrow (e.g. helper T lymphocytes, eosinophilic granulocytes). The background signals are more structured and indicate compartments of the haematopoietic and Lymphatic system without showing distinct lymph nodes or vessels which can be explained by the low resolution of 111-indium scintigrams (ca. 4 mm). The scintigrams of CD4/DR3 mice 24 and 48 hours after injection of the radiodiagnostic agent show a distinct reduction of the kidney signals resulting in an improvement of the representation of the spleen. The results indicate high proportions of human CD4-expressing cells. FACS analyses of CD4/DR3 mice confirm high proportions of human CD4-expressing helper T lymphocytes above all in the spleen [Laub 2001, JIM].

The differentiated signals indicate a selective accumulation of the radiodiagnostic agent in organs containing human CD4-expressing cells. This accumulation is specific since it does not occur in wild-type mice without a transgenic human CD4 as the target molecule and is due to the specific interaction between human CD4 and 111-indium-labelled anti-human CD4 Fab′ fragments.

### Example 7

### Specific Staining of Murine and Human Helper T Lymphocytes

This example shows that monoclonal antibodies to human and marine CD4 do not cross-react with one another.

Peripheral blood from humans and mice was incubated with fluorescein isothio-cyanate (FITC)-labelled monoclonal antibodies to murine CD4 (clone GK 1.5; cf. U.S. Pat. No. 6,146,614) and human CD4 (clone max. 16H5). In order to detect the T lymphocytes the cell suspensions were additionally incubated with phycoerythrin (PE)-labelled antibodies to murine or human CD3. After an incubation period of 30 min the cell suspensions were washed and analysed by flow cytometry (FACScan, Becton Dickinson).

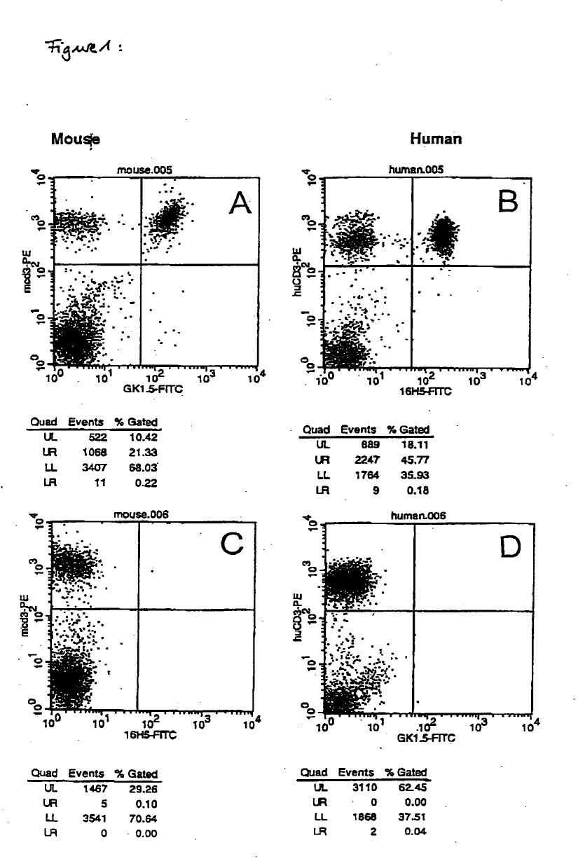

Neither CD3-positive nor CD3-negative cells were found in murine cell suspensions to which Max.16H5 also binds (quadrant UR and UL in blot C). Conversely no cells which bind GK. 1.5 (quadrants UR and UL in blot D) were found among the human cells. Hence the stainings in FIG. 1 show that GK 1.5 only binds to murine helper T lymphocytes (blot A) and that Max. 16H5 only binds to human helper T lymphocytes (blot B).

### Example 8

### Analysis of the Binding Site of Max. 16E15 (Anti-Human CD4) to Human CD4 (Epitope Mapping)

This example shows the identification of the Max. 16H5 binding site on the extracellular region of human CD4.

For this 177 peptides from the linear amino acid sequence of domains 1 to 4 (D1-D4) of the human CD4 molecule were synthesized on spots of a membrane derivatized with PEG spacers with the aid of an auto-spot robot (ASP222). The peptide sequences in the spots were 12 amino acids in length and each contained the 10 last amino acids of the previous sequence (pattern: 12mers with 10-er overlap).

These membranes were blocked with 2% milk powder and incubated for 120 min at room temperature with 10 μg/ml Max. 16H5. The membranes were subsequently washed and incubated for 75 mm at room temperature with a peroxidase-labelled goat anti-mouse antibody (ZAM-POD, Dianova). After a further washing step the ZAM-POD was detected with the aid of a chemoluminescent substrate (Super Signal Wes Dura, Pierce). The chemoluminescence signals on the membrane were detected and digitalized with the aid of a luminescence image reader (Fujifilm LAS-1000).

The following amino acid sequences were responsible for the binding of Max. 16H5 to the spotted peptides:

| | | |
|---|---|---|
| p34: | GDT VEL TCT ASQ | (SEQ ID NO. 1) |
| p46: | KKS IQF HWK K | (SEQ ID NO. 2) |
| p115: | KEE VQL LVF | (SEQ ID NO. 3) |
| p151: | PSV QCR SPR | (SEQ ID NO. 4) |
| p223: | FPL AFT VEK LTG SGE | (SEQ ID NO. 5) |
| p307: | KLII QEV NLV VMR | (SEQ ID NO. 6) |

The amino acid sequences used for the binding of Max. 16H5 was reconstructed from the positive spots on a three-dimensional model of human CD4. This showed that Max. 16H5 interacts with a spatially structured binding site (conformation epitope).

### Example 9

The following experiments were carried out under the direction and supervision of Prof. Emmrich as the inventor:

In an experimentally induced adjuvant arthritis in the rat

(1) Tc-99m-labelled anti-rat CD4 monoclonal antibodies and
(2) Tc-99m-labelled anti-rat CD4 monoclonal antibody FAB′ fragments
were compared with regard to their ability to give a sharp and contrasty image in the scintigram with a good signal-to-background ratio.

The scintigraphs were taken with a gamma camera. Subsequently the counts per time interval were determined for a predefined number of pixels in the scintigrams that were obtained.

This was carried out for two regions namely the arthritic joint (signal region) and a region on the lower leg of the rat which yielded a characteristic background signal.

The ratio between the two measured quantities “signal” and “background” was determined.

The following was found for whole anti-CD4 antibodies:

| | time after injection | | |
|---|---|---|---|
| | 1 hour | 2.5 hours | 4 hours |
| ankle/lower led ratio | 2.15 ± 0.15 | 2.50 ± 0.29 | 2.65 ± 0.41 |
| (signal-to-background | | | |
| ratio) | | | |

| | |
|---|---|
| average ratio over time (in | 2.29 ± 0.32 |
| saturation) | |

4 Hours after injecting the complete antibodies the ratio reached a stable saturation level and did not change again until the end of the experiment.

The following was found for the anti-CD4 Fab′ fragment:

| | time after injection | | |
|---|---|---|---|
| | 1 hour | 2.5 hours | 4 hours |
| ankle/lower led ratio | 2.48 ± 0.20 | 3.01 ± 0.19 | 3.15 ± 0.38 |
| (signal-to-background | | | |
| ratio) | | | |

| | |
|---|---|
| average ratio over time (in | 3.25 ± 0.39 |
| saturation) | |

After 4 hours the ratio also in this case reached a stable saturation level and remained unchanged until the end of the experiment (14 hours after injecting the fragments).

The anti-rat CD4 monoclonal antibody W3/25 (IgG1) was used as an antibody (or fragment thereof) in (1) and (2). It is directed against the equivalent of the human CD4 molecule in the rat on T helper cells and macrophages. The fragment was prepared from the whole antibody by pepsin digestion and purified on a protein A column, ultrafiltered and dialysed against PBS.

The radiolabelling was carried out by means of the mercaptoethanol method of Schwarz and Steinsträsser. The labelled immunoglobulin was administered to the anaesthetised rat by means of an i.v. catheter (200 μg, corresponding to 16 to 38 Mbq, Tc-labelled whole antibody/animal; 250 μg corresponding to ca. 37 Mbq, Tc-labelled fragments/animal.

The scintigram was recorded with the aid of a Siemens Basicam gamma camera equipped with a pinhole collimator.

1-25. (canceled)

26. A method of making a diagnostic agent for analyzing in vivo a migration or a distribution pattern of human CD4-bearing cells in a human subject, said method comprising the steps of:
obtaining a ligand having specificity for a human CD4 molecule, wherein the ligand is an antibody fragment or a recombinant antibody fragment, and wherein the antibody fragment is selected from the group consisting of: an F(ab′)₂ fragment, an Fab′ fragment, and an Fab fragment of the antibody Max.16H5, and wherein the recombinant antibody fragment is selected from the group consisting of: an F(ab′) fragment, an Fab′ fragment, and an Fab fragment of the antibody Max.16H5; and
attaching a label to the ligand, thereby forming a diagnostic agent.

27. The method of claim 26, wherein the ligand recognizes an amino acid sequence according to any of SEQ ID NOS.: 1-6, and selectively binds thereto.

28. The method of claim 26, wherein the antibody fragment or the recombinant antibody fragment recognizes a conformation epitope of human CD4 and bind thereto.

29. The method of claim 26, wherein the label is selected from the group consisting of: a gamma emitter, a positron emitter, a magnetic material, a density contrast material, and a combination thereof.

30. The method of claim 26, wherein the label of the ligand is a gamma emitter.

31. The method of claim 30, wherein the gamma emitter is technetium-99m or indium-111.

32. The method of claimed in claim 29, wherein the label of the ligand is a positron emitter which comprises one or more isotopes selected from the group consisting of fluorine-18, carbon-11, iodine-124, other isotopes for medical imaging methods and mixtures thereof.

33. The method of claimed in claim 29, wherein the label of the ligand comprises a magnetic material selected from the group consisting of gadolinium, superparamagnetic substances, hydrated iron oxide particles, other materials for medical imaging methods and mixtures thereof.

34. The method of claimed in claim 29, wherein the labeled ligand comprises a density contrast material.

35. The method of claim 26, wherein the human CD4-bearing cells are selected from the group consisting of: T lymphocytes, B lymphocytes, monocytes, macrophages, dendritic cells, Langerhans cells, and eosinophilic granulocytes.

36. The method of claim 35, wherein the lymphocytes are isolated from a cell population, wherein the cell population comprises non-lymphocytic cells.

37. The method of claim 26, wherein the cell population is derived from a human individual.

38. A composition containing a labeled ligand having specificity for the CD4 molecule and CD4-bearing cells or particles.

39. The composition as claimed in claim 38, wherein the CD4-bearing cells or particles are human CD4-bearing cells or particles.

40. The composition as claimed in claim 38 as a diagnostic agent.

41. A method for determining the extent and progression of diseases in which human CD4-bearing cells are of clinical importance comprising the steps:
(a) providing an in vivo analysis of the distribution and/or migration pattern of human CD4-bearing cells in an individual,
(b) providing a standard in vivo analysis of the distribution and/or migration pattern of human CD4-bearing cells in an individual and
(c) determining deviations of the analyses provided in step a) and step b) which enable the extent and progression of the disease to be determined.

42. The method as claimed in claim 41, wherein the disease is selected from the group consisting of autoimmune diseases, tumour diseases, infectious diseases and rejection crises after transplantations.

43. The method as claimed in claim 41, wherein the standard analysis in step b) is the analysis of the distribution and/or migration pattern of human CD4-bearing cells in a healthy individual.

44. The method as claimed in claim 41, wherein the standard analysis in step b) is a distribution and/or migration pattern of an individual which is obtained from a prior analysis of the same human individual.

45. The method as claimed in claim 41, wherein in step a) the analysis of the distribution and/or migration pattern of human CD4-bearing cells is provided in an individual who has been additionally treated for the disease.

46. The method as claimed in claim 45, wherein the treatment comprises administering a compound or a drug.

47. The method of claim 26, wherein the antibody fragment or the recombinant antibody fragment is derivatized.

48. The method of claim 26, wherein the antibody fragment or the recombinant antibody fragment is derivatized with a chelating agent.


SEQUENCE LISTING (6 sequences)

SEQ ID NO: 1 - protein, 12 aa, homo sapiens
GDTVELTCTASQ

SEQ ID NO: 2 - protein, 10 aa, homo sapiens
KKSIQFHWKK

SEQ ID NO: 3 - protein, 9 aa, homo sapiens
KEEVQLLVF

SEQ ID NO: 4 - protein, 9 aa, homo sapiens
PSVQCRSPR

SEQ ID NO: 5 - protein, 15 aa, homo sapiens
FPLAFTVEKLTGSGE

SEQ ID NO: 6 - protein, 13 aa, homo sapiens
KLIIQEVNLVVMR